(19) United States
(12) Patent Application Publication
(10) Pub. No.: US 2022/0259632 A1
(43) Pub. Date: Aug. 18, 2022

(54) METHOD OF OLIGONUCLEOTIDE SYNTHESIS

(71) Applicant: Nuclera Nucleics Ltd, Cambridge, Cambridgeshire (GB)

(72) Inventors: Michael Chun Hao Chen, Cambridge (GB); Gordon Ross McInroy, Cambridge (GB)

(21) Appl. No.: 17/436,314

(22) PCT Filed: Mar. 9, 2020
(86) PCT No.: PCT/GB2020/050558

§ 371 Date: Sep. 3, 2021

(30) Foreign Application Priority Data
Mar. 7, 2019 (GB) 1903055.0

(51) Int. Cl. C12P 19/34; B01J 19/00; C12N 9/12; C07H 21/04

(52) U.S. Cl. CPC C12P 19/34; B01J 19/0046; C12N 9/1264; C12Y 207/07031; B01J 2219/00722; B01J 2219/00596; B01J 2219/00608; B01J 2219/00713; B01J 2219/00716; C07H 21/04

(57) ABSTRACT

The invention relates to methods and kits for the synthesis of oligonucleotides via controlled, localised deprotection of 3′-ONH₂ groups on a solid support.

### FIELD OF THE INVENTION

The invention relates to methods and kits for the synthesis of oligonucleotides via controlled, localised deprotection of 3′-ONH₂ groups on a solid support.

### BACKGROUND OF THE INVENTION

Nucleic acid synthesis is vital to modern biotechnology. The rapid pace of development in the biotechnology arena has been made possible by the scientific community's ability to artificially synthesise DNA, RNA and proteins.

Artificial DNA synthesis allows biotechnology and pharmaceutical companies to develop a range of peptide therapeutics, such as insulin for the treatment of diabetes. It allows researchers to characterise cellular proteins to develop new small molecule therapies for the treatment of diseases our aging population faces today, such as heart disease and cancer. It even paves the way forward to creating life, as the Venter Institute demonstrated in 2010 when they placed an artificially synthesised genome into a bacterial cell.

However, current DNA synthesis technology does not meet the demands of the biotechnology industry. Despite being a mature technology, it is practically impossible to synthesise a DNA strand greater than 200 nucleotides in length, and most DNA synthesis companies only offer up to 120 nucleotides. In comparison, an average protein-coding gene is of the order of 2000-3000 contiguous nucleotides, a chromosome is at least a million contiguous nucleotides in length and an average eukaryotic genome numbers in the billions of nucleotides. In order to prepare nucleic acid strands thousands of base pairs in length, all major gene synthesis companies today rely on variations of a ‘synthesise and stitch’ technique, where overlapping 40-60-mer fragments are synthesised and stitched together by enzymatic copying and extension. Current methods generally allow up to 3 kb in length for routine production.

The reason DNA cannot be chemically synthesised beyond 120-200 nucleotides at a time is due to the current methodology for generating DNA, which uses synthetic chemistry (i.e., phosphoramidite technology) to couple a nucleotide one at a time to make DNA. Even if the efficiency of each nucleotide-coupling step is 99% efficient, it is mathematically impossible to synthesise DNA longer than 200 nucleotides in acceptable yields. The Venter Institute illustrated this laborious process by spending 4 years and 20 million USD to synthesise the relatively small genome of a bacterium.

Known methods of DNA sequencing use template-dependent DNA polymerases to add 3′-reversibly terminated nucleotides to a growing double-stranded substrate. In the ‘sequencing-by-synthesis’ process, each added nucleotide contains a dye, allowing the user to identify the exact sequence of the template strand. Albeit on double-stranded DNA, this technology is able to produce strands of between 500-1000 bps long. However, this technology is not suitable for de novo nucleic acid synthesis because of the requirement for an existing nucleic acid strand to act as a template.

Various attempts have been made to use a terminal deoxynucleotidyl transferase for de novo single-stranded DNA synthesis. Uncontrolled de novo single stranded DNA synthesis, as opposed to controlled, takes advantage of TdT's deoxynucleoside triphosphate (dNTP) 3′ tailing properties on single-stranded DNA to create, for example, homopolymeric adaptor sequences for next-generation sequencing library preparation. In controlled extensions, a reversible deoxynucleoside triphosphate termination technology needs to be employed to prevent uncontrolled addition of dNTPs to the 3′-end of a growing DNA strand. The development of a controlled single-stranded DNA synthesis process through TdT would be invaluable to in situ DNA synthesis for gene assembly or hybridization microarrays as it removes the need for an anhydrous environment and allows the use of various polymers incompatible with organic solvents. However, TdT has not been shown to efficiently add nucleoside triphosphates containing 3′-O-reversibly terminating moieties for building up a nascent single-stranded DNA chain necessary for a de novo synthesis cycle, and thus the synthesis of long strands is inefficient.

Oligonucleotides can be synthesized either individually or on an array. In flow based array DNA synthesis systems, it is necessary to selectively deprotect a defined set of synthesis sites. This has previously been achieved through means such as light-mediated deprotection and masks, as well as electrochemical generation of acid and patterned electrodes. However the synthesis relies on organic solvents and requires a number of washing steps changes of reagent per monomer addition.

There is therefore a need for a new method to efficiently prepare large numbers of oligonucleotides in order to provide an improved method of nucleic acid synthesis that is able to overcome the problems associated with currently available methods.

### BRIEF DESCRIPTION OF THE FIGURES

FIG. 1. SEQ ID 728 after incubation with addition solution containing an engineered TdT and 3′-ONH₂ dNTP. The mass spectra deconvolutes to give an observed mass of 4838.76. The expected mass following addition is 4836.81.

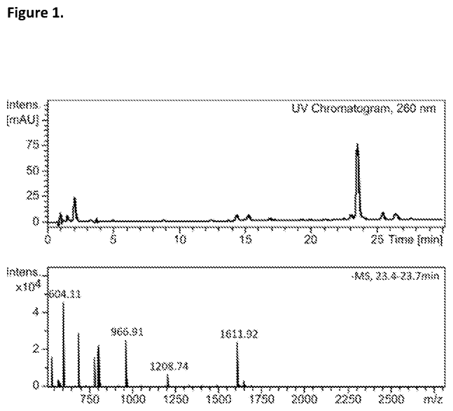

FIG. 2. The effect of changing pH on the efficiency of nitrite-mediated 3′-aminoxy deprotection. As the pH is raised the extent of aminoxy to hydroxyl conversion within 5 minutes falls significantly.

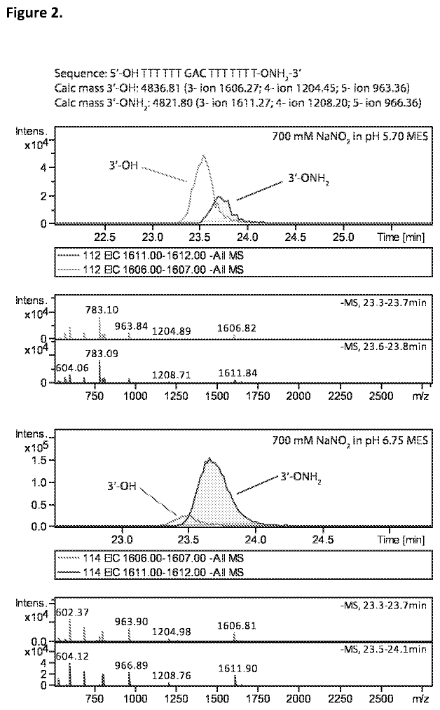

### SUMMARY OF THE INVENTION

The invention relates to methods and kits for the synthesis of oligonucleotides via controlled, localised deprotection of 3′-ONH₂ groups on a solid support. The inventors have appreciated that the nitrite-mediated deprotection of the 3′-O-aminoxy reversible terminator shows pH dependence, which can therefore be used to locally deprotect the 3′-O-aminoxy reversible terminator from desired regions of a solid support.

Disclosed is a method for the synthesis of a plurality of immobilised nucleic acids of differing sequence, comprising:
a. taking a system with a solid support having a plurality of 5′-end immobilised nucleic acids which are 3′-ONH₂ protected and a nitrite deprotection solution that is inactive at the basal pH of the system;
b. lowering the pH at a site localized to one or more selected immobilised nucleic acids, thereby activating the deprotection solution to deprotect the 3′-ends of a subset of the immobilised nucleic acids;
c. extending the deprotected 3′-ends of the immobilized nucleic acids;
d. lowering the pH at a site localized to one or more selected immobilised nucleic acids, thereby activating the deprotection solution to deprotect the 3′-ends of a subset of the immobilised nucleic acids, wherein the localized sites are different to those of step b;
e. extending the deprotected 3′-ends of the immobilized nucleic acids, thereby synthesizing a plurality of immobilised nucleic acids of differing sequence.

Disclosed is a method for the synthesis of a plurality of immobilised nucleic acids of differing sequence, comprising:
a. taking a system with a solid support having a plurality of 5′-end immobilised nucleic acids which are 3′-ONH₂ protected and a nitrite deprotection solution that is inactive at the basal pH of the system;
b. lowering the pH at a site localized to one or more selected immobilised nucleic acids, thereby activating the deprotection solution to deprotect the 3′-ends of a subset of the immobilised nucleic acids;
c. extending the deprotected 3′-ends of the immobilized nucleic acids;
d. repeating steps b-c with desired subsets of immobilized nucleic acid, thereby synthesizing a plurality of immobilized nucleic acids of differing sequence.

The nitrite solution can optionally be present during the cycles of extension, providing the pH of the extension solution is above the level where deprotection occurs. This reduces the number of reagent exchanges. The system can comprise nucleotides with 3′-ONH₂ protection, an optionally modified terminal transferase enzyme (TdT), buffer components to retain a basal pH and a nitrite deprotection solution that is inactive at the basal pH.

Thus disclosed is a method for the synthesis of a plurality of immobilised nucleic acids of differing sequence, comprising:
a. taking a system with a solid support having a plurality of 5′-end immobilised nucleic acids which are 3′-ONH₂ protected and a nitrite deprotection solution that is inactive at the basal pH of the system;
b. lowering the pH at a site localized to one or more selected immobilised nucleic acids, thereby activating the deprotection solution to deprotect the 3′-ends of a subset of the immobilised nucleic acids;
c. extending the deprotected 3′-ends of the immobilized nucleic acids using nucleotides with 3′-ONH₂ protection and an optionally modified terminal transferase enzyme (TdT);
d. lowering the pH at a site localized to one or more selected immobilised nucleic acids, thereby activating the deprotection solution to deprotect the 3′-ends of a subset of the immobilised nucleic acids, wherein the localized sites are different to those of step b;
e. extending the deprotected 3′-ends of the immobilized nucleic acids using nucleotides with 3′-ONH₂ protection and an optionally modified terminal transferase enzyme (TdT), thereby synthesizing a plurality of immobilised nucleic acids of differing sequence.

Alternatively the extension and deprotection solutions can be separate. If the solutions are separate, disclosed is a method comprising the steps of
a. taking a system with a solid support having a plurality of 5′-end immobilised nucleic acids which are 3′-ONH₂ protected;
b. adding a nitrite deprotection solution that is inactive at the basal pH of the system;
c. lowering the pH at a site localized to one or more selected immobilised nucleic acids, thereby activating the deprotection solution to deprotect the 3′-ends of a subset of the immobilised nucleic acids;
d. removing the nitrite deprotection solution;
e. extending the deprotected 3′-ends of the immobilized nucleic acids;
f. adding a nitrite deprotection solution that is inactive at the basal pH of the system;
g. lowering the pH at a site localized to one or more selected immobilised nucleic acids, thereby activating the deprotection solution to deprotect the 3′-ends of a subset of the immobilised nucleic acids, wherein the localized sites are different to those of step c;
h. extending the deprotected 3′-ends of the immobilized nucleic acids, thereby synthesizing a plurality of immobilised nucleic acids of differing sequence.

Again the extension can be performed using nucleotides with 3′-ONH₂ protection and an optionally modified terminal transferase enzyme (TdT). The method can comprise the steps of
a. taking a system with a solid support having a plurality of 5′-end immobilised nucleic acids which are 3′-ONH₂ protected;
b. adding a nitrite deprotection solution that is inactive at the basal pH of the system;
c. lowering the pH at a site localized to one or more selected immobilised nucleic acids, thereby activating the deprotection solution to deprotect the 3′-ends of a subset of the immobilised nucleic acids;
d. removing the nitrite deprotection solution;
e. extending the deprotected 3′-ends of the immobilized nucleic acids using nucleotides with 3′-ONH₂ protection and an optionally modified terminal transferase enzyme (TdT);
f. adding a nitrite deprotection solution that is inactive at the basal pH of the system;
g. lowering the pH at a site localized to one or more selected immobilised nucleic acids, thereby activating the deprotection solution to deprotect the 3′-ends of a subset of the immobilised nucleic acids, wherein the localized sites are different to those of step c;
h. extending the deprotected 3′-ends of the immobilized nucleic acids using nucleotides with 3′-ONH₂ protection and an optionally modified terminal transferase enzyme (TdT), thereby synthesizing a plurality of immobilised nucleic acids of differing sequence.

Disclosed is a method for the synthesis of a plurality of immobilised nucleic acids of differing sequence, comprising:
a. taking a system with a solid support having a plurality of 5′-end immobilised nucleic acids which are 3′-ONH₂ protected;
b. adding a nitrite deprotection solution that is inactive at the basal pH of the system;
c. lowering the pH at a site or sites localized to one or more selected immobilised nucleic acids, thereby activating the deprotection solution to deprotect the 3′-ends of a subset of the immobilised nucleic acids;
d. removing the nitrite deprotection solution;
e. extending the deprotected 3′-ends of the immobilized nucleic acids;
f. repeating steps b-e with desired subsets of immobilized nucleic acid, thereby synthesizing a plurality of immobilized nucleic acids of differing sequence.

Generally each extension cycle contains a single species of nucleotide. The nucleotide varies cycle by cycle in order to build up the desired sequences at the different locations. Thus a different nucleotide solution is added compared to the previous cycle of extension, and the solutions are repeated in cycles to grow differing sequences in differing areas of the solid support.

The immobilised nucleic acids can be single stranded DNA species or double stranded DNA species, with a 3′ overhang, or a mixture thereof.

The pH can be controlled by a variety of means, including an electrochemically generated acid (EGA) or photogenerated acid. The EGA can be selected from the electrolysis of water or the modulation of a hydroquinone/benzoquinone system. It will be apparent to the person skilled in the art that any means of selectively changing the pH in a localized area can be used in the disclosed method.

When the solution contains nucleotides with 3′-ONH₂ protection, an optionally modified terminal transferase enzyme (TdT), buffer components to retain a basal pH and a nitrite deprotection solution that is inactive at the basal pH, the modified TdT is active at the basal pH of the system and generally inactive at the altered pH required for deprotection of the 3′-ends of the immobilised nucleic acids, thereby preventing extension of the released OH groups prior to addition of the next nucleotide.

If a homopolymer sequence is desired, it may be possible to prepare the sequence simply by altering the pH of a solution containing nucleotides with 3′-ONH₂ protection, an optionally modified terminal transferase enzyme (TdT), buffer components to retain a basal pH and a nitrite deprotection solution that is inactive at the basal pH. In such cases the modified TdT is active at the basal pH of the system and inactive at the altered pH required for deprotection of the 3′-ends of the immobilised nucleic acids. Thus deprotection only occurs when the pH is lowered, and extension of the freed OH groups occurs when the pH is buffered back to the basal level.

In order for efficient deprotection, the altered pH required for deprotection of the 3′-ends of the immobilised nucleic acids is pH 5.5 or lower.

In order to ensure no deprotection occurs, the basal pH of the system is 7.5 or higher. Generally the nitrite solution is buffered. The buffer can be selected from MES, citrate, phosphate, acetate or a combination thereof. The buffer concentration can be 0.1-5000 mM, preferably between 500 mM and 2500 mM.

The concentration of nitrite can be 500 mM or higher. The concentration of nitrite can be 700 mM or higher. The concentration of nitrite can be 500 -1000mM. The nitrite can be sodium nitrite.

In order to improve generation of localized acid, the system can comprise alternating anodic and cathodic electrodes.

The nucleotides grown can be of any desired length. Each of the plurality of immobilized nucleic acids can be extended by at least 25 bases.

After production, the oligonucleotide sequences can be released from being immobilized, for example by cleavage of the group attaching the oligonucleotide to the support.

Disclosed is a method for the selective deprotection of immobilised nucleic acids, comprising:
a. taking a system comprising:
   i. a solid support wherein the solid support has a plurality of immobilised nucleic acids which are 3′-ONH₂ protected;
   ii. a nitrite deprotection solution that is inactive at the basal pH of the system; and
b. temporarily lowering the pH at a site localized to one or more selected immobilised nucleic acids, thereby activating the deprotection solution to deprotect the 3′-ends of a subset of the immobilised nucleic acids.

Disclosed is a kit for preparing a plurality of immobilised nucleic acids of differing sequence, comprising:
a. a solid support having a plurality of 5′-end immobilised nucleic acids which are 3′-ONH₂ protected;
b. a buffered nitrite deprotection solution that is inactive at the basal pH of the system;
c. nucleotides with 3′-ONH₂ protection; and
d. an optionally modified terminal transferase enzyme (TdT).

### DETAILED DESCRIPTION OF THE INVENTION

In flow based array DNA synthesis systems, it is necessary to selectively deprotect a defined set of synthesis sites. This has previously been achieved through means such as light-mediated deprotection and masks, as well as electrochemical generation of acid and patterned electrodes.

While previous systems have used electrochemically generated acid (EGA) to remove the DMT protecting group in phosphoramidite oligonucleotide synthesis, it is the EGA-mediated pH change that directly causes acid-mediated removal of the DMT group. In this embodiment, the EGA-mediated pH change modulates the kinetics of a secondary reaction—in this case the nitrite-mediated conversion of the aminoxy moiety (—ONH2) to the hydroxyl moiety (—OH).

Selective deprotection can be achieved in a system where all sites are exposed to nitrite solution at pH 6-9, preferably above pH 7.5 (or another pH where nitrite-mediated deprotection of aminoxy nucleotides does not occur), and a defined set of sites have their pH changed through (EGA). This EGA-mediated pH change would be to a pH suitable for nitrite-mediated deprotection of the aminoxy group—such as pH 5.50 for example. EGA can be achieved through electrolysis of water, or the through modulation of electroactive agents such as the hydroquinone/benzoquinone redox pair.

Ideally EGA only affects the defined sites where EGA occurs; diffusion of EGA could lead to synthesis errors due to the deprotection of 3′-O reversible terminators on non-specified sites. The presence of a buffered nitrite solution would help reduce the diffusion of EGA away from the electrode, as while the EGA would exceed the buffering capacity near the electrode, it would not exceed the buffering capacity at a distance from the electrode. The buffering demands (ie: concentration of buffer) will be related to the concentration of electroactive agents if they are used in an EGA system. Another mechanism to reduce errors caused by diffusion of EGA from the electrode involves alternating anodic and cathodic electrodes, as acid is generated at one and consumed at the other. The electrodes can be patterned such that synthesis sites of one polarity are isolated from other synthesis sites by electrodes of the other polarity.

It is possible to envisage a system where all reaction components are present in the same mixture.

For example a solution could contain:

Engineered TdT

Reversibly terminated nucleotide

Pyrophosphatase (optional)

Buffer (at pH 7.5 for example)

Secondary buffering system (optional)

Sodium nitrite

Quinine/hydroquinone (optional)

In the absence of EGA, this solution acts as an addition solution. The nitrite is inactive at the chosen pH (e.g. 7.5) while the engineered TdT is active and performs addition of reversibly terminated nucleotides to single stranded DNA.

In the presence of EGA, this solution acts as a deblocking solution. The nitrite is active at acidic pH (e.g. pH 5.5 and below) while the engineered TdT is inactive and unable to perform nucleotide incorporation.

Such a system would reduce the number of wash steps necessary in a synthesis process. Such a system would have utility in the rapid switching between addition and deblocking modes. For example, where the enzyme recovers functionality following a pH 7.5->pH 5.5->pH 7.5 cycle.

A dual buffer system may be used to control the pH change upon production of EGA. With a single buffer system, once the buffering capacity is overcome it is possible the pH may rapidly drop to highly acidic pH such as pH 1-3. With a dual buffer system, a low concentration primary buffer with a pKa near the addition pH would resist change induced by EGA, but quickly be overcome. A secondary buffer at a higher concentration with a pKa near the desired pH for deblocking (e.g. 5-5.5) would then strongly resist further decreases in pH and prevent highly acidic pH being reached. As DNA suffers increasing damage with decreasing pH, a dual buffer system offers an advantage. An alternative to a dual buffer system would be to control the change in pH through limiting the time EGA is generated.

The inventors have previously developed a selection of engineered terminal transferase enzymes, any of which may be used in the current process.

Terminal transferase enzymes are ubiquitous in nature and are present in many species. Many known TdT sequences have been reported in the NCBI database http://www.ncbi.nlm.nih.gov/. The sequences of the various described terminal transferases show some regions of highly conserved sequence, and some regions which are highly diverse between different species.

The inventors have modified the terminal transferase from Lepisosteus oculatus TdT (spotted gar) (shown below). However the corresponding modifications can be introduced into the analagous terminal transferase sequences from any other species, including the sequences listed above in the various NCBI entries. The amino acid sequence of the spotted gar (Lepisosteus oculatus) is shown below (SEQ ID no 1):

MLHIPIFPPIKKRQKLPESRNSCKYEVKFSEVAIFLVERKMGSSRRKFLTN
LARSKGFRIEDVLSDAVTHVVAEDNSADELWQWLQNSSLGDLSKIEVLDIS
WFTECMGAGKPVQVEARHCLVKSCPVIDQYLEPSTVETVSQYACQRRTTME
NHNQIFTDAFAILAENAEFNESEGPCLAFMRAASLLKSLPHAISSSKDLEG
LPCLGDQTKAVIEDILEYGQCSKVQDVLCDDRYQTIKLFTSVFGVGLKTAE
KWYRKGFHSLEEVQADNAIHFTKMQKAGFLYYDDISAAVCKAEAQAIGQIV
EETVRLIAPDAIVTLIGGFRRGKECGHDVDFLITTPEMGKEVWLLNRLINR
LQNQGILLYYDIVESTFDKTRLPCRKFEAMDHFQKCFAIIKLKKELAAGRV
QKDWKAIRVDFVAPPVDNFAFALLGWTGSRQFERDLRRFARHERKMLLDNH
ALYDKTKKIFLPAKTEEDIFAHLGLDYIDPWQRNA

The inventors have identified various regions in the amino acid sequence having improved properties. Certain regions improve the solubility and handling of the enzyme. Certain other regions improve the ability to incorporate nucleotides with modifications at the 3′-position.

Modifications which improve the solubility include a modification within the amino acid region WLLNRLINRLQNQGILLYYDIV shown highlighted in the sequence below.

MLHIPIFPPIKKRQKLPESRNSCKYEVKFSEVAIFLVERKMGSSRRKFLTNLARSKGFRIEDVLSDAV
THVVAEDNSADELWQWLQNSSLGDLSKIEVLDISWFTECMGAGKPVQVEARHCLVKSCPVIDQYLEPS
TVETVSQYACQRRTTMENHNQIFTDAFAILAENAEFNESEGPCLAFMRAASLLKSLPHAISSSKDLEG
LPCLGDQTKAVIEDILEYGQCSKVQDVLCDDRYQTIKLFTSVFGVGLKTAEKWYRKGFHSLEEVQADN
AIHFTKMQKAGFLYYDDISAAVCKAEAQAIGQIVEETVRLIAPDAIVTLTGGFRRGKECGHDVDFLIT

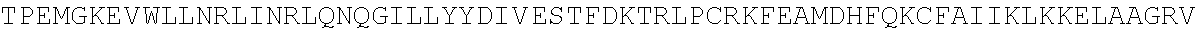

QKDWKAIRVDFVAPPVDNFAFALLGWTGSRQFERDLRRFARHERKMLLDNHALYDKTKKIFLPAKTEE
DIFAHLGLDYIDPWQRNA

Modifications which improve the incorporation of modified nucleotides can be at one or more of selected regions shown below. The second modification can be selected from one or more of the amino acid regions VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP shown highlighted in the sequence below.

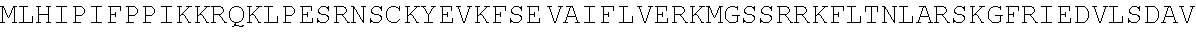

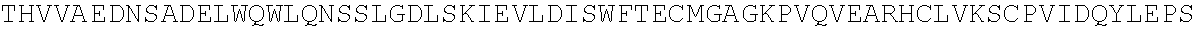

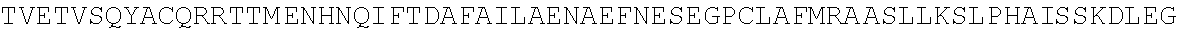

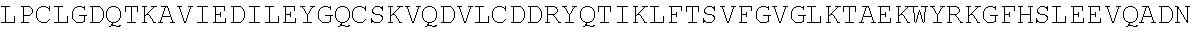

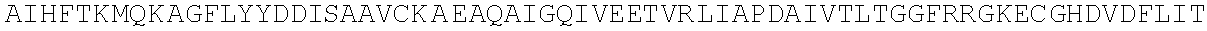

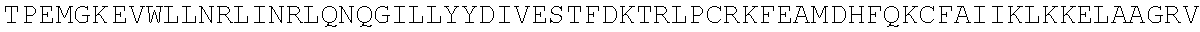

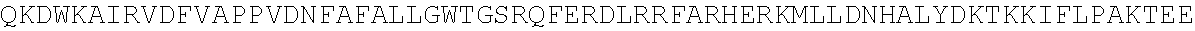

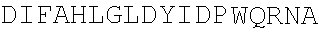

Described herein is a modified terminal deoxynucleotidyl transferase (TdT) enzyme comprising at least one amino acid modification when compared to a wild type sequence SEQ ID NO 1 or the homologous amino acid sequence of a terminal deoxynucleotidyl transferase (TdT) enzyme in other species, wherein the modification is selected from one or more of the amino acid regions WLLNRLINRLQNQGILLYYDI, VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species.

The terminal transferase or modified terminal transferase can be any enzyme capable of template independent strand extension. The enzyme may be a modified terminal deoxynucleotidyl transferase (TdT) enzyme comprising amino acid modifications when compared to a wild type sequence Lepisosteus oculatus TdT (spotted gar) sequence or a truncated version thereof or the homologous amino acid sequence of a terminal deoxynucleotidyl transferase (TdT) enzyme in other species or the homologous amino acid sequence of Polμ, Polβ, Polλ, and Polθ of any species or the homologous amino acid sequence of X family polymerases of any species, wherein the amino acid is modified at one or more of the amino acids:

V32, A33, I34, F35, A53, V68, V71, E97, I101, M108, G109, A110, Q115, V116, S125, T137, Q143, M152, E153, N154, H155, N156, Q157, I158, I165, N169, N173, S175, E176, G177, P178, C179, L180, A181, F182, M183, R184, A185, L188, H194, A195, I196, S197, S198, S199, K200, E203, G204, D210, Q211, T212, K213, A214, I216, E217, D218, L220, Y222, V228, D230, Q238, T239, L242, L251, K260, G261, F262, H263, S264, L265, E267, Q269, A270, D271, N272, A273, H275, F276, T277, K278, M279, Q280, K281, S291, A292, A293, V294, C295, K296, E298, A299, Q300, A301, Q304, I305, T309, V310, R311, L312, I313, A314, I318, V319, T320, G328, K329, E330, C331, L338, T341, P342, E343, M344, G345, K346, W349, L350, L351, N352, R353, L354, I355, N356, R357, L358, Q359, N360, Q361, G362, I363, L364, L365, Y366, Y367, D368, I369, V370, K376, T377, C381, K383, D388, H389, F390, Q391, K392, F394, I397, K398, K400, K401, E402, L403, A404, A405, G406, R407, D411, A421, P422, P423, V424, D425, N426, F427, A430, R438, F447, A448, R449, H450, E451, R452, K453, M454, L455, L456, D457, N458, H459, A460, L461, Y462, D463, K464, T465, K466, K467, T474, D477, D485, Y486, I487, D488, P489.

The enzyme may be a modified terminal deoxynucleotidyl transferase (TdT) enzyme comprising at least one amino acid modification when compared to a wild type sequence or a truncated version thereof, wherein the modification is selected from one or more of the amino acid regions WLLNRLINRLQNQGILLYYDIV, VAIF, MGA, MENHNQI, SEGPCLAFMRA, HAISSS, DQTKA, KGFHS, QADNA, HFTKMQK, SAAVCK, EAQA, TVRLI, GKEC, TPEMGK, DHFQK, LAAG, APPVDNF, FARHERKMLLDNHALYDKTKK, and DYIDP of the sequence of Lepisosteus oculatus TdT (spotted gar) or the homologous regions in other species or the homologous regions of Polμ, Polβ, Polλ, and Polθ of any species or the homologous regions of X family polymerases of any species.

Homologous refers to protein sequences between two or more proteins that possess a common evolutionary origin, including proteins from superfamilies in the same species of organism as well as homologous proteins from different species. Such proteins (and their encoding nucleic acids) have sequence homology, as reflected by their sequence similarity, whether in terms of percent identity or by the presence of specific residues or motifs and conserved positions. A variety of protein (and their encoding nucleic acid) sequence alignment tools may be used to determine sequence homology. For example, the Clustal Omega multiple sequence alignment program provided by the European Molecular Biology Laboratory (EMBL) can be used to determine sequence homology or homologous regions. To aid alignment comparison sequences of the enzymes from Bos Taurus (cow) and Mus musculus (mouse) are shown in SEQ ID NOs 2 and 3.

Improved sequences as described herein can contain both modifications, namely

a. a first modification is within the amino acid region WLLNRLINRLQNQGILLYYDI of the sequence of SEQ ID NO 1 or the homologous region in other species; and

b. a second modification is selected from one or more of the amino acid regions VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species.

Disclosed is a modified terminal deoxynucleotidyl transferase (TdT) enzyme comprising at least one amino acid modification when compared to a wild type sequence SEQ ID NO 1 or the homologous amino acid sequence of a terminal deoxynucleotidyl transferase (TdT) enzyme in other species, wherein the modification is selected from one or more of the amino acid regions WLLNRLINRLQNQGILLYYDI, VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species.

Further disclosed is a modified terminal deoxynucleotidyl transferase (TdT) enzyme comprising at least two amino acid modifications when compared to a wild type sequence SEQ ID NO 1 or the homologous amino acid sequence of a terminal deoxynucleotidyl transferase (TdT) enzyme in other species, wherein;

a. a first modification is within the amino acid region WLLNRLINRLQNQGILLYYDIV of the sequence of SEQ ID NO 1 or the homologous region in other species; and

b. a second modification is selected from one or more of the amino acid regions VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species.

For the purposes of brevity, the modifications are further described in relation to SEQ ID NO 1, but the modifications are applicable to the sequences from other species, for example those sequences listed above having sequences in the NCBI database.

The modification within the region WLLNRLINRLQNQGILLYYDIV or the corresponding region from other species help improve the solubility of the enzyme. The modification within the amino acid region WLLNRLINRLQNQGILLYYDIV can be at one or more of the underlined amino acids.

Particular changes can be selected from W-Q, N-P, R-K, L-V, R-L, L-W, Q-E, N-K, Q-K or I-L.

The sequence WLLNRLINRLQNQGILLYYDIV can be altered to QLLPKVINLWEKKGLLLYYDLV.

The second modification improves incorporation of nucleotides having a modification at the 3′ position in comparison to the wild type sequence. The second modification can be selected from one or more of the amino acid regions VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species. The second modification can be selected from two or more of the amino acid regions VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species shown highlighted in the sequence below.

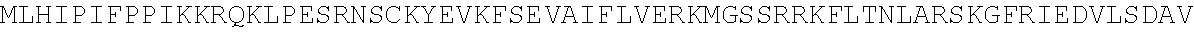

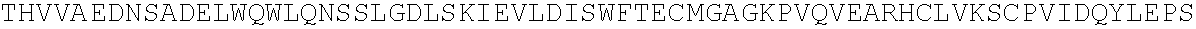

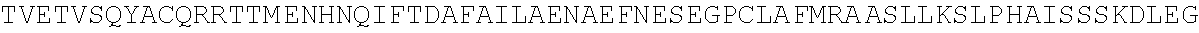

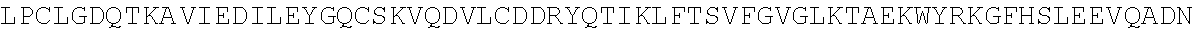

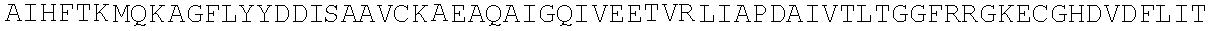

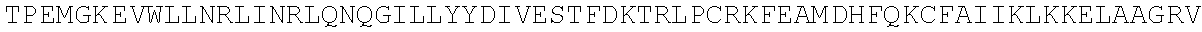

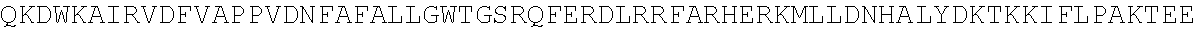

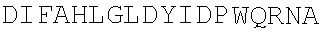

The identified positions commence at positions V32, E74, M108, F182, T212, D271, M279, E298, A421, L456, Y486. Modifications disclosed herein contain at least one modification at the defined positions.

The modified amino acid can be in the region FM RA. The modified amino acid can be in the region QADNA. The modified amino acid can be in the region EAQA. The modified amino acid can be in the region APP. The modified amino acid can be in the region LDNHA. The modified amino acid can be in the region YIDP. The region FARHERKMLLDNHA is advantageous for removing substrate biases in modifications. The FARHERKMLLDNHA region appears highly conserved across species.

The modification selected from one or more of the amino acid regions FMRA, QADNA, EAQA, APP, FARHERKMLLDNHA, and YIDP can be at the underlined amino acid(s).

The positions for modification can include A53, V68, V71, D75, E97, I101, G109, Q115, V116, S125, T137, Q143, N154, H155, Q157, I158, I165, G177, L180, A181, M183, A195, K200, T212, K213, A214, E217, T239, F262, S264, Q269, N272, A273, K281, S291, K296, Q300, T309, R311, E330, T341, E343, G345, N352, N360, Q361, I363, Y367, H389, L403, G406, D411, A421, P422, V424, N426, R438, F447, R452, L455, and/or D488.

Amino acid changes include any one of A53G, V68I, V71I, D75N, D75Q, E97A, I101V, G109E, G109R, Q115E, V116I, V116S, S125R, T137A, Q143P, N154H, H155C, Q157K, Q157R, 1158M, 1165V, G177D, L180V, A181E, M183R, A195P, K200R, T212S, K213S, A214R, E217Q, T239S, F262L, S264T, Q269K, N272K, A273S, A273T, K281R, S291N, K296R, Q300D, T309A, R311W, E330N, T341S, E343Q, G345R, N352Q, N360K, Q361K, I363L, Y367C, H389A, L403R, G406R, D411N, A421L, A421M, A421V, P422A, P422C, V424Y, N426R, R438K, F447W, R452K, L455I, and/or D488P.

Amino acid changes include any two or more of A53G, V68I, V71I, D75N, D75Q, E97A, I101V, G109E, G109R, Q115E, V116I, V116S, S125R, T137A, Q143P, N154H, H155C, Q157K, Q157R, 1158M, 1165V, G177D, L180V, A181E, M183R, A195P, K200R, T212S, K213S, A214R, E217Q, T239S, F262L, S264T, Q269K, N272K, A273S, A273T, K281R, S291N, K296R, Q300D, T309A, R311W, E330N, T341S, E343Q, G345R, N352Q, N360K, Q361K, I363L, Y367C, H389A, L403R, G406R, D411N, A421L, A421M, A421V, P422A, P422C, V424Y, N426R, R438K, F447W, R452K, L455I, and/or D488P.

The modification of QADNA to KADKA, QADKA, KADNA, QADNS, KADNT, or QADNT is advantageous for the incorporation of 3′-O-modified nucleoside triphosphates to the 3′-end of nucleic acids and removing substrate biases during the incorporation of modified nucleoside triphosphates. The modification of APPVDN to MCPVDN, MPPVDN, ACPVDR, VPPVDN, LPPVDR, ACPYDN, LCPVDN, or MAPVDN is advantageous for the incorporation of 3′-O-modified nucleoside triphosphates to the 3′-end of nucleic acids and removing substrate biases during the incorporation of modified nucleoside triphosphates. The modification of FARHERKMLLDRHA to WARHERKMILDNHA, FARHERKMILDNHA, WARHERKMLLDNHA, FARHERKMLLDRHA, or FARHEKKMLLDNHA is also advantageous for the incorporation of 3′-O-modified nucleoside triphosphates to the 3′-end of nucleic acids and removing substrate biases during the incorporation of modified nucleoside triphosphates.

The modification can be selected from one or more of the following sequences FRRA, QADKA, EADA, MPP, FARHERKMLLDRHA, and YIPP. Included is a modified terminal deoxynucleotidyl transferase (TdT) enzyme wherein the second modification is selected from two or more of the following sequences FRRA, QADKA, EADA, MPP, FARHERKMLLDRHA, and YIPP. Included is a modified terminal deoxynucleotidyl transferase (TdT) enzyme wherein the second modification contains each of the following sequences FRRA, QADKA, EADA, MPP, FARHERKMLLDRHA, and YIPP.

In order to aid purification of the expressed sequence, the amino acid can be further modified. For example the amino acid sequence can contain one or more further histidine residues at the terminus. Included is a modified terminal deoxynucleotidyl transferase (TdT) enzyme comprising any one of SEQ ID NOs 4 to 173 or a truncated version thereof. Sequences 4-173 are the full length sequences derived from the spotted gar. Included is a modified terminal deoxynucleotidyl transferase (TdT) enzyme comprising any one of SEQ ID NOs 174 to 343. Sequences 174 to 343 are N-truncated sequences as spotted gar/bovine chimeras. Sequences 344 to 727 are spotted Gar sequences in truncated form. Additionally, for these sequences, there is an N-terminal sequence that is incorporated simply as a protease cleavage site (MENLYFQG . . . ).

References herein to ‘nucleoside triphosphates’ refer to a molecule containing a nucleoside (i.e. a base attached to a deoxyribose or ribose sugar molecule) bound to three phosphate groups. Examples of nucleoside triphosphates that contain deoxyribose are: deoxyadenosine triphosphate (dATP), deoxyguanosine triphosphate (dGTP), deoxycytidine triphosphate (dCTP) or deoxythymidine triphosphate (dTTP). Examples of nucleoside triphosphates that contain ribose are: adenosine triphosphate (ATP), guanosine triphosphate (GTP), cytidine triphosphate (CTP) or uridine triphosphate (UTP). Other types of nucleosides may be bound to three phosphates to form nucleoside triphosphates, such as naturally occurring modified nucleosides and artificial nucleosides.

Therefore, references herein to ‘3′-blocked nucleoside triphosphates’ refer to nucleoside triphosphates (e.g., dATP, dGTP, dCTP or dTTP) which have an additional group on the 3′ end which prevents further addition of nucleotides, i.e., by replacing the 3′-OH group with a protecting group with a group 3′-ONH₂.

In one embodiment, the nitrite cleaving agent is added in the presence of a cleavage solution comprising a denaturant, such as urea, guanidinium chloride, formamide or betaine. The addition of a denaturant has the advantage of being able to disrupt any undesirable secondary structures in the DNA. In a further embodiment, the cleavage solution comprises one or more buffers. It will be understood by the person skilled in the art that the choice of buffer is dependent on the exact cleavage chemistry and cleaving agent required.

References herein to an ‘initiator sequence’ refer to a short oligonucleotide with a free 3′-end which the 3′-blocked nucleoside triphosphate can be attached to. In one embodiment, the initiator sequence is a DNA initiator sequence. In an alternative embodiment, the initiator sequence is an RNA initiator sequence.

References herein to a ‘DNA initiator sequence’ refer to a small sequence of DNA which the 3′-blocked nucleoside triphosphate can be attached to, i.e., DNA will be synthesised from the end of the DNA initiator sequence.

In one embodiment, the initiator sequence is between 5 and 50 nucleotides long, such as between 5 and 30 nucleotides long (i.e. between 10 and 30), in particular between 5 and 20 nucleotides long (i.e., approximately 20 nucleotides long), more particularly 5 to 15 nucleotides long, for example 10 to 15 nucleotides long, especially 12 nucleotides long.

In one embodiment, the initiator sequence is single-stranded. In an alternative embodiment, the initiator sequence is double-stranded. It will be understood by persons skilled in the art that a 3′-overhang (I.e., a free 3′-end) allows for efficient addition.

In one embodiment, the initiator sequence is immobilised on a solid support. This allows TdT and the cleaving agent to be removed without washing away the synthesised nucleic acid. The initiator sequence may be attached to a solid support stable under aqueous conditions so that the method can be easily performed via a flow setup.

In one embodiment, the initiator sequence is immobilised on a solid support via a reversible interacting moiety, such as a chemically-cleavable linker, an antibody/immunogenic epitope, a biotin/biotin binding protein (such as avidin or streptavidin), or glutathione-GST tag. Therefore, in a further embodiment, the method additionally comprises extracting the resultant nucleic acid by removing the reversible interacting moiety in the initiator sequence, such as by incubating with proteinase K.

In one embodiment, the initiator sequence contains a base or base sequence recognisable by an enzyme. A base recognised by an enzyme, such as a glycosylase, may be removed to generate an abasic site which may be cleaved by chemical or enzymatic means. A base sequence may be recognised and cleaved by a restriction enzyme.

In a further embodiment, the initiator sequence is immobilised on a solid support via a chemically-cleavable linker, such as a disulfide, allyl, or azide-masked hemiaminal ether linker. Therefore, in one embodiment, the method additionally comprises extracting the resultant nucleic acid by cleaving the chemical linker through the addition of tris(2-carboxyethyl)phosphine (TCEP) or dithiothreitol (DTT) for a disulfide linker; palladium complexes or an allyl linker; or TCEP for an azide-masked hemiaminal ether linker.

In one embodiment, the resultant nucleic acid is extracted and amplified by polymerase chain reaction using the nucleic acid bound to the solid support as a template. The initiator sequence could therefore contain an appropriate forward primer sequence and an appropriate reverse primer could be synthesised.

In one embodiment, the terminal deoxynucleotidyl transferase (TdT) of the invention is added in the presence of an extension solution comprising one or more buffers (e.g., Tris or cacodylate), one or more salts (e.g., Na⁺, K⁺, Mg²⁺, Mn²⁺, Cu²⁺, Zn²⁺, Co²⁺, etc. all with appropriate counterions, such as Cl) and inorganic pyrophosphatase (e.g., the Saccharomyces cerevisiae homolog). It will be understood that the choice of buffers and salts depends on the optimal enzyme activity and stability. The use of an inorganic pyrophosphatase helps to reduce the build-up of pyrophosphate due to nucleoside triphosphate hydrolysis by TdT. Therefore, the use of an inorganic pyrophosphatase has the advantage of reducing the rate of (1) backwards reaction and (2) TdT strand dismutation.

Also disclosed is a kit comprising a terminal deoxynucleotidyl transferase (TdT) as defined herein in combination with:

a. a solid support having a plurality of 5′-end immobilised nucleic acids which are 3′-ONH₂ protected;

b. a buffered nitrite deprotection solution that is inactive at the basal pH of the system; and

c. nucleotides with 3′-ONH₂ protection, and the modified terminal transferase enzyme (TdT)

### Exemplary Process

1. Have an array of immobilised single stranded DNA species, or double stranded DNA species with a 3′ overhang, where the 3′ base has a 3′-ONH₂ moiety. For example, this array may be patterned on to a surface or may be through deposition of beads.

2. Expose all immobilised DNA sites to inactive nitrite deprotection solution (the 3′-ONH₂ moiety remains intact at all locations).

3. Selectively change the pH at a subset of immobilised DNA sites. The pH may be changed by generating acid through electrochemical or photochemical means. Where the pH is changed, active nitrite deprotection solution is produced. Active nitrite solution converts the 3′-ONH₂ moiety to a 3′-hydroxyl moiety.

4. Cease generation of acid.

5. Expose all immobilised DNA sites to addition solution containing (reversibly terminated) nucleotides, a terminal transferase enzyme, buffer components and optionally a pyrophosphatase. Only those sites exposed to active nitrite deprotection solution will contain the 3′-hydroxyl moiety that is permissive for enzyme-mediated incorporation of a nucleotide (which may be reversibly terminated).

6. Repeat steps 1-5 (with optional wash steps in between) to generate an array of oligonucleotides with pre-defined and independent sequences.

The sequences grow at different lengths in different places on the support, depending on the presence or absence of the blocking ONH₂.

### Exemplary Data

SEQ ID 728: TTTTTTGACTTTTTT

Exact Molecular Weight: 4517.75

Seq 728 was incubated at 37° C. for 20 minutes with addition solution containing engineered TdT and 3′-ONH₂ dNTP. The reaction was stopped by heating to 80° C. for 5 minutes and the oligonucleotide purified by gel filtration. The oligonucleotide was then incubated with nitrite solution at various pH values for 5 minutes before being quenched and purified by gel filtration. Oligonucleotides were analysed by LCMS (Buffer A: 100 mM HFIP, 10 mM TEA in water; Buffer B: methanol).

Peak separation between the 3′-hydroxyl and 3′-aminoxy species is minimal, so data was analysed by extracted mass. Deprotection was found to be complete at pH 5.2 and 5.5, i.e. only 3′-hydroxyl species was detected and there was an absence of 3′-aminoxy species. At pH 5.7 there was significant 3′-aminoxy remaining. At pH 6.75 only 9% 3′-hydroxyl was detected to 91% 3′-aminoxy—indicating a much reduced deprotection efficiency.

1. A method for the synthesis of a plurality of immobilised nucleic acids of differing sequence, comprising:
a. taking a system with a solid support having a plurality of 5′-end immobilised nucleic acids which are 3′-ONH2 protected and a nitrite deprotection solution that is inactive at the basal pH of the system;
b. lowering the pH at a site localized to one or more selected immobilised nucleic acids, thereby activating the deprotection solution to deprotect the 3′-ends of a subset of the immobilised nucleic acids;
c. extending the deprotected 3′-ends of the immobilized nucleic acids using nucleotides with 3′-ONH2 protection and an optionally modified terminal transferase enzyme (TdT);
d. lowering the pH at a site localized to one or more selected immobilised nucleic acids, thereby activating the deprotection solution to deprotect the 3′-ends of a subset of the immobilised nucleic acids, wherein the localized sites are different to those of step b;
e. extending the deprotected 3′-ends of the immobilized nucleic acids using nucleotides with 3′-ONH2 protection and an optionally modified terminal transferase enzyme (TdT), thereby synthesizing a plurality of immobilised nucleic acids of differing sequence.

2. The method of claim 1 comprising the steps of
a. taking a system with a solid support having a plurality of 5′-end immobilised nucleic acids which are 3′-ONH2 protected;
b. adding a nitrite deprotection solution that is inactive at the basal pH of the system;
c. lowering the pH at a site localized to one or more selected immobilised nucleic acids, thereby activating the deprotection solution to deprotect the 3′-ends of a subset of the immobilised nucleic acids;
d. removing the nitrite deprotection solution;
e. extending the deprotected 3′-ends of the immobilized nucleic acids using nucleotides with 3′ -ONH2 protection and an optionally modified terminal transferase enzyme (TdT);
f. adding a nitrite deprotection solution that is inactive at the basal pH of the system;
g. lowering the pH at a site localized to one or more selected immobilised nucleic acids, thereby activating the deprotection solution to deprotect the 3′-ends of a subset of the immobilised nucleic acids, wherein the localized sites are different to those of step c;
h. extending the deprotected 3′-ends of the immobilized nucleic acids using nucleotides with 3′ -ONH₂ protection and an optionally modified terminal transferase enzyme (TdT), thereby synthesizing a plurality of immobilised nucleic acids of differing sequence.

3. The method of claim 1 wherein a different nucleotide solution is added compared to the previous cycle of extension, and the solutions are repeated in cycles to grow differing sequences in differing areas of the solid support.

4. The method of claim 1, wherein the immobilised nucleic acids are single stranded DNA species or double stranded DNA species, with a 3′ overhang, or a mixture thereof.

5. The method of claim 1, wherein the pH change is the result of an electrochemically generated acid (EGA).

6. The method of claim 5, wherein the method used to generate the EGA is selected from: the electrolysis of water or the modulation of a hydroquinone/benzoquinone system.

7. The method of claim 1, wherein the pH change is the result of a photogenerated acid.

8. The method of claim 1, wherein the modified TdT is active at the basal pH of the system and inactive at the altered pH required for deprotection of the 3′-ends of the immobilised nucleic acids.

9. The method of claim 1, wherein the altered pH required for deprotection of the 3′-ends of the immobilised nucleic acids is pH 5.5 or lower.

10. The method of claim 9, wherein basal pH of the system is 7.5 or higher.

11. The method of claim 1, wherein the nitrite solution is buffered.

12. The method of claim 11, wherein the buffer is selected from MES, citrate, phosphate, acetate or a combination thereof.

13. The method according to claim 11 wherein the concentration of buffer is between 500 mM and 2500 mM.

14. The method of claim 1 wherein the nitrite is present at a concentration of between 500-1000 mM.

15. The method of claim 1 wherein the nitrite is sodium nitrite.

16. The method of claim 1, wherein the system comprises alternating anodic and cathodic electrodes.

17. The method of claim 1, wherein each of the plurality of immobilized nucleic acids is extended by at least 25 bases.

18. The method of claim 1, wherein the oligonucleotide sequences are released from being immobilized.

19. A method for the selective deprotection of immobilised nucleic acids, comprising:
a. taking a system comprising:
i. a solid support wherein the solid support has a plurality of immobilised nucleic acids which are 3′-ONH₂ protected;
ii. a nitrite deprotection solution that is inactive at the basal pH of the system; and
b. temporarily lowering the pH at a site localized to one or more selected immobilised nucleic acids, thereby activating the deprotection solution to deprotect the 3′-ends of a subset of the immobilised nucleic acids.

20. A kit for preparing a plurality of immobilised nucleic acids of differing sequence, comprising:
a. a solid support having a plurality of 5′-end immobilised nucleic acids which are 3′-ONH₂ protected;
b. a buffered nitrite deprotection solution that is inactive at the basal pH of the system;
c. nucleotides with 3′-ONH₂ protection; and
d. an optionally modified terminal transferase enzyme (TdT).
